(19) United States
(12) Patent Application Publication
(10) Pub. No.: US 2026/0034182 A1
(43) Pub. Date: Feb. 5, 2026

(54) POSTBIOTIC EXTRACT OF LACTOBACILLUS PLANTARUM CB102 AND METHODS FOR PREPARING THE SAME AND REDUCING BLOOD URIC ACID LEVEL USING THE SAME

(71) Applicant: CHAMBIO CO., LTD., Taichung City (TW)

(72) Inventors: Hung-Pin CHIU, Taichung City (TW); Yu-Ting LIN, Taichung City (TW)

(21) Appl. No.: 19/079,955

(22) Filed: Mar. 14, 2025

(30) Foreign Application Priority Data
Aug. 5, 2024 (TW) 113129230

(51) Int. Cl. A61K 35/747; A61P 13/12

(52) U.S. Cl. CPC A61K 35/747; A61P 13/12

(57) ABSTRACT

A postbiotic extract of Lactobacillus plantarum CB102 is prepared by the steps of: subjecting the Lactobacillus plantarum CB102 to a protein extraction treatment, so as to obtain a protein extract; and subjecting the protein extract to a hydrolysis treatment with a protease, so as to obtain the postbiotic extract of Lactobacillus plantarum CB102. The Lactobacillus plantarum CB102 is deposited at the Leibniz Institute DSMZ-German Collection of Microorganisms and Cell Cultures GmbH under an accession number DSM 33894 in accordance with the Budapest Treaty. A method for preparing a postbiotic extract of Lactobacillus plantarum CB102, and methods for reducing a blood uric acid level and alleviating a uric acid metabolism-associated disorder using the postbiotic extract of Lactobacillus plantarum CB102 are also provided.

### CROSS-REFERENCE TO RELATED APPLICATION

This application claims priority to Taiwanese Invention patent application No. 113129230, filed on Aug. 5, 2024, the entire disclosure of which is incorporated by reference herein.

### FIELD

The present disclosure relates to a postbiotic extract of Lactobacillus plantarum CB102 and a method for preparing the same. The disclosure also relates to methods for reducing a blood uric acid level and alleviating a uric acid metabolism-associated disorder using the postbiotic extract of Lactobacillus plantarum CB102.

### BACKGROUND

Uric acid is a final oxidation product of purine catabolism via xanthine oxidase (XO), and is excreted from a body through kidneys in urine. Various factors, including diet (e.g., excessive intake of purine-rich foods), environment, medications, and genetics may lead to abnormalities in uric acid metabolism (including overproduction or underexcretion of uric acid), such that a blood uric acid level is excessively elevated, which in turn increases a risk of uric acid metabolism-associated disorders, such as gout, hyperuricemia, etc.

Due to changes in modern dietary habits, the incidence and prevalence of the uric acid metabolism-associated disorders have been increasing year by year, with a growing trend of patients becoming younger. Medications currently used to reduce the blood uric acid level include uric acid synthesis inhibitors and uricosuric agents. However, in clinical practice, use of the aforesaid medications may incur problems, such as limited effectiveness and a high likelihood of occurrence of side effects.

Lactic acid bacteria (LAB) are conferred with the generally recognized as safe (GRAS) status, and are widely known and commonly used as probiotics. Common genera of LAB include Lactobacillus spp., Lactococcus spp., Pediococcus spp., Enterococcus spp., Streptococcus spp., Bifidobacterium spp., Bacillus spp., Leuconostoc spp., etc.

Previous studies have demonstrated that certain strains of LAB are capable of reducing the blood uric acid level so as to alleviate hyperuricemia. For example, Y. Li et al. (2022), Front. Nutr., doi: 10.3389/fnut.2022.993951. discloses that administration of live Lacticaseibacillus rhamnosus 1155 or/and Limosilactobacillus fermentum 2644 can reduce blood uric acid levels in hyperuricemia rats, whereas administration of heat-killed Lacticaseibacillus rhamnosus 1155 or Limosilactobacillus fermentum 2644 has no effect on blood uric acid levels in hyperuricemia rats. In addition, it has been reported, in J. Cao et al. (2022), Front. Nutr., doi: 10.3389/fnut.2022. 954545, that administration of live Lactobacillus plantarum Q7 can reduce blood uric acid levels in hyperuricemia rats.

In view of the aforesaid, there is still a need to develop an effective LAB-related matter that can exhibit satisfactory efficacy in reducing a blood uric acid level and alleviating a uric acid metabolism-associated disorder.

### SUMMARY

Therefore, in a first aspect, the present disclosure provides a postbiotic extract of Lactobacillus plantarum CB102, which can alleviate at least one of the drawbacks of the prior art. The postbiotic extract of Lactobacillus plantarum CB102 is prepared by the steps of: subjecting the Lactobacillus plantarum CB102 to a protein extraction treatment, so as to obtain a protein extract; and subjecting the protein extract to a hydrolysis treatment with a protease, so as to obtain the postbiotic extract of Lactobacillus plantarum CB102. The Lactobacillus plantarum CB102 is deposited at the Leibniz Institute DSMZ-German Collection of Microorganisms and Cell Cultures GmbH under an accession number DSM 33894 in accordance with the Budapest Treaty.

In a second aspect, the present disclosure provides a method for reducing a blood uric acid level, which can alleviate at least one of the drawbacks of the prior art, and which includes administering to a subject in need thereof a composition including the aforesaid postbiotic extract of Lactobacillus plantarum CB102.

In a third aspect, the present disclosure provides a method for alleviating a uric acid metabolism-associated disorder, which can alleviate at least one of the drawbacks of the prior art, and which includes administering to a subject in need thereof a composition including the aforesaid postbiotic extract of Lactobacillus plantarum CB102.

In a fourth aspect, the present disclosure provides a method for preparing a postbiotic extract of Lactobacillus plantarum CB102, which can alleviate at least one of the drawbacks of the prior art, and which includes: subjecting the Lactobacillus plantarum CB102 to a protein extraction treatment, so as to obtain a protein extract, and subjecting the protein extract to a hydrolysis treatment with a protease, so as to obtain the postbiotic extract of Lactobacillus plantarum CB102. The Lactobacillus plantarum CB102 is deposited at the Leibniz Institute DSMZ-German Collection of Microorganisms and Cell Cultures GmbH under an accession number DSM 33894 in accordance with the Budapest Treaty.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the ultra-high performance liquid chromatography coupled with tandem mass spectrometry (UHPLC-MS/MS) spectrum of the postbiotic extract of the present disclosure in Example 2, infra.

FIG. 2 shows the change in blood uric acid level determined over time in each group of mice in Example 4, infra, in which: the English letters indicate the results of statistical analyses; if any two groups have the same English letter, there is no significant difference between the two groups; and if any two groups do not have the same English letter, there is a significant difference between the two groups (p<0.05).

FIG. 3 shows the blood urea nitrogen (BUN) level determined in each group of mice in Example 4, infra, in which: the English letters indicate the results of statistical analyses; if any two groups have the same English letter, there is no significant difference between the two groups; and if any two groups do not have the same English letter, there is a significant difference between the two groups (p<0.05).

FIG. 4 shows the blood creatinine (Cr) level determined in each group of mice in Example 4, infra, in which: the English letters indicate the results of statistical analyses; if any two groups have the same English letter, there is no significant difference between the two groups; and if any two groups do not have the same English letter, there is a significant difference between the two groups (p<0.05).

FIG. 5 shows the activity of xanthine oxidase (XO) in blood determined in each group of mice in Example 4, infra, in which: the English letters indicate the results of statistical analyses; if any two groups have the same English letter, there is no significant difference between the two groups; and if any two groups do not have the same English letter, there is a significant difference between the two groups (p<0.05).

FIG. 6 shows the activity of xanthine oxidase (XO) in liver determined in each group of mice in Example 4, infra, in which: the English letters indicate the results of statistical analyses; if any two groups have the same English letter, there is no significant difference between the two groups; and if any two groups do not have the same English letter, there is a significant difference between the two groups (p<0.05).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a postbiotic extract of Lactobacillus plantarum CB102, which is prepared by the steps of: subjecting the Lactobacillus plantarum CB102 to a protein extraction treatment, so as to obtain a protein extract; and subjecting the protein extract to a hydrolysis treatment with a protease, so as to obtain the postbiotic extract of Lactobacillus plantarum CB102. The Lactobacillus plantarum CB102 is deposited at the Leibniz Institute DSMZ-German Collection of Microorganisms and Cell Cultures GmbH under an accession number DSM 33894 in accordance with the Budapest Treaty.

The present disclosure also provides a method for preparing a postbiotic extract of Lactobacillus plantarum CB102, which includes: subjecting the Lactobacillus plantarum CB102 to a protein extraction treatment, so as to obtain a protein extract; and subjecting the protein extract to a hydrolysis treatment with a protease, so as to obtain the postbiotic extract of Lactobacillus plantarum CB102. The Lactobacillus plantarum CB102 is deposited at the Leibniz Institute DSMZ-German Collection of Microorganisms and Cell Cultures GmbH under an accession number DSM 33894 in accordance with the Budapest Treaty.

As used herein, the term “postbiotic extract” can be used interchangeably with other term such as “hydrolysate of microbial protein extract”, and means that the postbiotic extract is prepared by subjecting a postbiotic (e.g., non-viable microbial cells, microbial fractions, and cell lysates) to a degradation process (e.g., an enzymatic hydrolysis treatment or a solvent extraction treatment). The postbiotic extract may include metabolites, proteins, lipids, carbohydrates, vitamins, organic acids, or cell wall components of microorganisms.

According to the present disclosure, the Lactobacillus plantarum CB102 used to prepare the postbiotic extract may be viable or non-viable, concentrated or non-concentrated, a liquid, a paste, a semi-solid, or a solid (e.g., a pellet, a granule, or a powder), and may be heat-inactivated, frozen, dried, freeze-dried, or spray/fluid bed-dried.

According to the present disclosure, the protein extraction treatment may be carried out using techniques well-known to those skilled in the art. In certain embodiments, the protein extraction treatment includes the steps of subjecting a cell sample to a cell wall lysis process, followed by conducting a protein precipitation process and then a protein separation process. The procedures and operating conditions for the aforesaid steps are within the expertise and routine skills of those skilled in the art, with reference to journal articles, e.g., P. J. Tian et al. (2015), Int. J. Mol. Sci., 16(8): 20033-20049.

According to the present disclosure, the protease may be selected from the group consisting of a glutamic acid protease, a serine protease, a glutamyl endopeptidase, an exopeptidase, and combinations thereof. In certain embodiments, the protease is the glutamic acid protease.

According to the present disclosure, the protease may be obtained as a commercial product, or may be prepared using techniques well-known to those skilled in the art.

According to the present disclosure, the hydrolysis treatment may be carried out using techniques well-known to those skilled in the art.

It should be noted that the operating conditions for the hydrolysis treatment can be adjusted based on factors such as a weight ratio of the protein extract to the protease, so as to achieve an optimal hydrolysis result. A selection of these operating conditions for the hydrolysis treatment can be routinely determined by those skilled in the art.

According to the present disclosure, a weight ratio of the protein extract to the protease ranges from 1.0:0.001 to 1.0:0.1. In certain embodiments, the weight ratio of the protein extract to the protease is 1.0:0.01.

According to the present disclosure, the hydrolysis treatment may be conducted at a temperature ranging from 40° C. to 60° C. for a time period ranging from 1 hour to 2 hours. In an exemplary embodiment, the hydrolysis treatment is conducted at 50° C. for 2 hours.

The present disclosure also provides a method for reducing a blood uric acid level, which includes administering to a subject in need thereof a composition including the aforesaid postbiotic extract of Lactobacillus plantarum CB102. The present disclosure also provides a method for alleviating a uric acid metabolism-associated disorder, which includes administering to a subject in need thereof a composition including the aforesaid postbiotic extract of Lactobacillus plantarum CB102.

According to the present disclosure, the uric acid metabolism-associated disorder may be selected from the group consisting of gout, hyperuricemia, gouty arthritis, kidney disease, urolithiasis, nephrolithiasis, and combinations thereof.

According to the present disclosure, the uric acid metabolism-associated disorder may further include complications associated with a high blood uric acid level, such as diabetes mellitus and a cardiovascular disease.

According to the present disclosure, alleviation of the uric acid metabolism-associated disorder may include at least one of the following: reducing a blood uric acid level, reducing blood uremic toxin levels (e.g., blood urea nitrogen (BUN) and creatinine (Cr)), inhibiting uric acid production, suppressing expression of xanthine oxidase (XO), and inhibiting an activity of xanthine oxidase (XO).

As used herein, the term “administering” can be used interchangeably with other term such as “administration”, and means introducing, providing or delivering a pre-determined ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

According to the present disclosure, the composition may further include a xanthine oxidase (XO) inhibitor, e.g., allopurinol.

According to the present disclosure, the composition may be formulated as a food product using a standard technique well known to one of ordinary skill in the art. For example, the composition may be formulated in the form of a food additive, which is added to an edible material to prepare a food product for human or animal consumption.

As used herein, the term “food product” refers to any article or substance that can be ingested by a subject into the body thereof. Examples of the food product may include, but are not limited to, milk powders, fermented milk, yogurt, butter, beverages (e.g., tea, coffee, etc.), functional beverages, a flour product, baked foods, confectionery, candies, fermented foods, animal feeds, health foods, and dietary supplements.

According to the present disclosure, the food product may further include a food additive widely employed in the art of food-manufacturing. Examples of the food additive may include, but are not limited to, a starch, a dextrin, lactose, maize flours, rice flours, tricalcium phosphate, silicon dioxide, magnesium stearate, calcium carbonate, sucrose, glucose, fructose, a sugar alcohol, an oligosaccharide, a sugar substitute, fruit juice powders, yeast powders, skim milk powders, casein, whey protein, soybean protein, amino acid, citric acid, citrate, lactic acid, lactate and nucleotide.

According to the present disclosure, the composition may be prepared in the form of a pharmaceutical composition. The pharmaceutical composition may be formulated into a suitable dosage form for oral or parenteral administration using technology well known to those skilled in the art.

According to the present disclosure, examples of the dosage form suitable for oral administration may include, but are not limited to, sterile powders, tablets, troches, lozenges, pellets, capsules, dispersible powders or granules, solutions, suspensions, emulsions, syrup, elixir, slurry, and the like. In certain embodiments, the pharmaceutical composition is formulated into a capsule dosage form.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intramuscular injection, intravenous injection, intraarterial injection, intraepidermal injection, subcutaneous injection, intradermal injection, and intralesional injection. In certain embodiments, the pharmaceutical composition is formulated into a dosage form suitable for intravenous injection.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the dose and frequency of administration of the postbiotic extract of Lactobacillus plantarum CB102 may vary depending on the following factors: the severity of the illness or disorder to be treated, routes of administration, and weight, age, physical condition and response of the subject to be treated. In general, the postbiotic extract of Lactobacillus plantarum CB102 may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials

### 1. Lactic Acid Bacterial (LAB) Strains

Lactobacillus plantarum CB102, which is known and readily available to the public and which is disclosed in TW I808582 B, was obtained from the Microbiology Research Laboratory of the Department of Food Science and Biotechnology, National Chung-Hsing University, Taichung, Taiwan, and has been deposited at the Bioresource Collection and Research Center (BCRC) of the Food Industry Research and Development Institute (FIRDI) (No. 331, Shih-Pin Rd., Hsinchu City 300, Taiwan). In addition, Lactobacillus plantarum CB102 has also been deposited under the terms of the Budapest Treaty on the International Recognition of the Deposit of Microorganisms for the Purpose of Patent Procedure at the International Depositary Authority, i.e., Leibniz Institute DSMZ-German Collection of Microorganisms and Cell Cultures GmbH (Inhoffenstr. 7B, D-38124 Braunschweig, Germany) in accordance with the Budapest Treaty.

The relevant information regarding Lactobacillus plantarum CB102 (including accession number and date of deposit) is listed in Table 1 below.

TABLE 1

| LAB strain | Accession number | Date of deposit |
|---|---|---|
| Lactobacillus plantarum | BCRC 910893 | May 08, 2019 |
| CB102 | DSM 33894 | Jun. 09, 2021 |

In addition, eight LAB strains used as comparative strains and sources thereof are listed in Table 2 below.

TABLE 2

| | LAB strain | |
|---|---|---|
| Genus | (accession number) | Source |
| Limosilactobacillus | Limosilactobacillus | Isolated from |
| | fermentum CECT5716 | breast milk of |
| | | healthy human |
| Lactobacillus | Lactobacillus rhamnosus | Isolated from |
| | GG (LGG) | intestine of |
| | | healthy human |
| | Lactobacillus acidophilus | Microbiology |
| | JCM1132 | Research |
| | (BCRC 10695; DSM 20079) | Laboratory of |
| | Lactobacillus casei | Department of |
| | JCM1134 | Food Science |
| | (BCRC 10697; DSM 20011) | and |
| Bifidobacterium | Bifidobacterium bifidum | Biotechnology, |
| | JCM1255 | National |
| | (BCRC 11845; DSM 20456) | Chung-Hsing |
| | Bifidobacterium lactis | University, |
| | JCM10602 | Taichung, |
| | (BCRC 17394; DSM 10140) | Taiwan |
| | Bifidobacterium longum | |
| | CB108 | |
| | (BCRC 910894; DSM 33895) | |
| Bacillus | Bacillus coagulans CB85 | |
| | (BCRC 911010; DSM 33893) | |

### 2. Medium for Culturing LAB Stains

The medium for culturing the nine LAB strains described in section 1 of “General Experimental Materials” was prepared using the recipe shown in Table 3 below.

TABLE 3

| Ingredient | Amount (g/L) |
|---|---|
| Peptone | 10 |
| Yeast extract | 5 |
| Beef extract | 10 |
| Glucose | 20 |
| Potassium hydrogen phosphate | 2 |
| Sodium acetate | 5 |
| Ammonium citrate | 2 |
| Magnesium sulfate | 0.2 |
| Manganese sulfate | 0.05 |
| Tween 80 | 1 |

The remainder was pure water

### 3. Experimental Mice

Male ICR mice (6 weeks old, with a body weight of approximately 35 g) used in the following experiments were purchased from BioLasco Taiwan Co., Ltd. All the experimental mice were housed in an animal room with an independent air conditioning system under the following laboratory conditions: an alternating 12-hour light and 12-hour dark cycle, a temperature maintained at 23±2° C., and a relative humidity maintained at 60±10%. The mice were provided with water and fed ad libitum. All experimental procedures involving the experimental mice were in compliance with the legal provision of the National Institutes of Health (NIH), USA, and were carried out according to the Guide for the Care and Use of Laboratory Animals.

### General Procedures:

### 1. Statistical Analysis

All the experiments described below were performed in triplicates. The experimental data of all the test groups are expressed as mean±standard error of the mean (SEM), and were analyzed using Tukey's range test, so as to evaluate the differences between the groups. Statistical significance is indicated by p<0.05.

### Example 1. Preparation of Postbiotic Extract of Lactobacillus plantarum CB102

First, the Lactobacillus plantarum CB102 described in section 1 of “General Experimental Materials” was inoculated in the medium prepared by the recipe shown in Table 3 above, followed by cultivation in an incubator at 37° C. for 18 hours, so as to obtain a bacterial solution of Lactobacillus plantarum CB102.

Next, the bacterial solution of Lactobacillus plantarum CB102 was subjected to a protein extraction treatment that included the following steps (a) to (c). In step (a), the bacterial solution of Lactobacillus plantarum CB102 was subjected to a centrifugation treatment at 10,000 rpm and 25° C. for 15 minutes, followed by pouring off the resultant cell culture supernatant, so as to collect the resultant cell pellet. In step (b), the resultant cell pellet was mixed with 1 mg/mL of lysozyme (Manufacturer: BioVision, Cat. no.: 8005) at a weight ratio of 1:99 so as to allow bacterial cell walls of Lactobacillus plantarum CB102 to be hydrolyzed at a temperature of 37° C. for 1 hour, followed by conducting a homogenization treatment using a sonicator (Manufacturer: SCICO, Model no.: SC-HM100), so as to obtain a homogeneous substance. In step (c), the homogeneous substance was subjected to a protein precipitation treatment in accordance with an ammonium sulfate precipitation method well-known to those skilled in the art, followed by conducting a dialysis treatment using a dialysis membrane (Manufacturer: Spectra/Por Biotech, Cat. no.: 132720) to remove ammonium sulfate, so as to obtain a protein extract of Lactobacillus plantarum CB102.

Thereafter, the protein extract of Lactobacillus plantarum CB102 was mixed with a glutamic acid protease (Manufacturer: MedChemExpress, Cat. no.: HY-P2935) at a weight ratio of 1.0:0.01, followed by conducting an enzymatic hydrolysis reaction at a temperature of 50° C. and a pH value of 7 for 2 hours, so as to obtain the postbiotic extract of Lactobacillus plantarum CB102. Afterwards, the postbiotic extract of Lactobacillus plantarum CB102 was subjected to a freeze-drying treatment or a spray-drying treatment, so as to obtain a freeze-dried/spray-dried powder of the postbiotic extract of Lactobacillus plantarum CB102 (abbreviated as postbiotic extract of the present disclosure).

### Example 2. Ultra-High Performance Liquid Chromatography Coupled with Tandem Mass Spectrometry (UHPLC-MS/MS) Analysis

### Experimental Procedures:

The postbiotic extract of the present disclosure prepared in Example 1 was dissolved in an appropriate amount of deionized water, thereby obtaining a test sample of the postbiotic extract of the present disclosure having a concentration of 20 mg/mL.

The test sample was subjected to ultra-high performance liquid chromatography coupled with tandem mass spectrometry (UHPLC-MS/MS) analysis using technology well-known to those skilled in the art. The operating parameters and conditions for performing UHPLC-MS/MS are summarized in Table 4 below.

TABLE 4

| | |
|---|---|
| UHPLC-MS/MS | UltiMate 3000 HPLC system (Manufacturer: |
| instrument | Dionex, Model no.: UltiMate 3000) equipped |
| | with a LTQ-Orbitrap mass spectrometer |
| | (Manufacturer: Thermo Fisher Scientific, Model |
| | no.: LTQ Orbitrap XL) |
| Type of | Gemini 3 μm NX-C18 110 Å Column |
| chromatography | (Manufacturer: Phenomenex, Model no.: 00F- |
| column | 4453-E0) |
| Size of | Length: 150 mm; |
| chromatography | inner diameter: 4.6 mm |
| column | |
| Detection | 214 nm |
| wavelength | |
| Detection mass- | 150 m/z to 2000 m/z |
| to charge ratio | |
| Mobile phase | 70% acetonitrile including 0.1% (w/w) |
| | trifluoroacetic acid |
| Flow rate of | 30 μL/min |
| test sample | |

### Results:

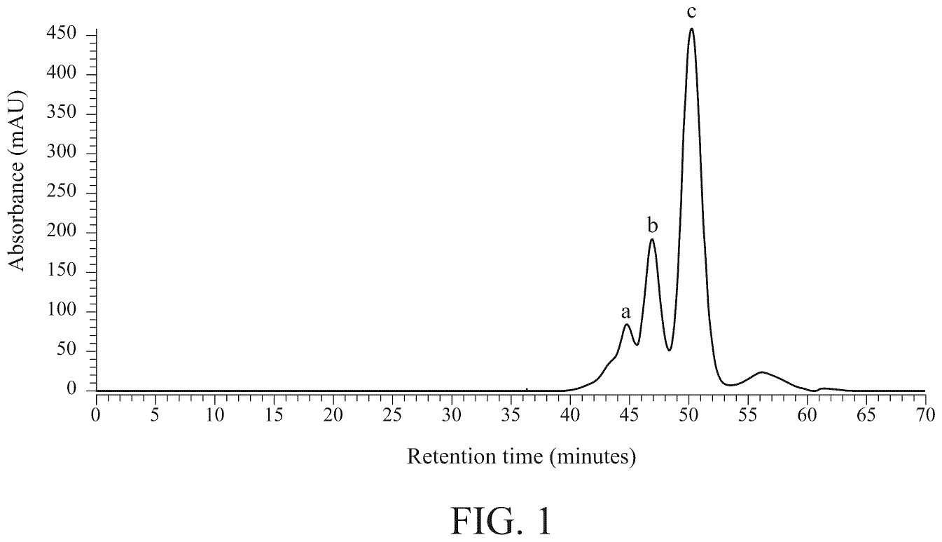

FIG. 1 shows the UHPLC-MS/MS spectrum of the postbiotic extract of the present disclosure. As shown in FIG. 1, three main peaks (i.e., peaks a, b, and c) were observed during a retention time from 0 minutes to 70 minutes, indicating the presence of three major components in the postbiotic extract of the present disclosure.

### Example 3. Evaluation of the Effect of Postbiotic Extract of Lactobacillus plantarum CB102 on Inhibiting Activity of Xanthine Oxidase (XO) In Vitro

In this example, in order to understand whether the postbiotic extract of the present disclosure has an effect of reducing uric acid production in vitro, the postbiotic extract of the present disclosure prepared in Example 1 was subjected to determination of xanthine oxidase inhibition rate in vitro.

### Experimental Materials:

### 1. Preparation of Postbiotic Extracts of Eight Comparative Strains

A freeze-dried/spray-dried powder of the postbiotic extract of a respective one of the eight comparative strains shown in Table 2 above was prepared according to the procedures described in Example 1. To be specific, the thus obtained freeze-dried/spray-dried powder of the postbiotic extract of the respective one of the eight comparative strains was abbreviated as postbiotic extract of the respective one of the eight comparative strains.

### 2. Preparation of Test Solution

An appropriate amount of a respective one of the protein extract of Lactobacillus plantarum CB102 prepared in Example 1, the postbiotic extract of the present disclosure prepared in Example 1, and the postbiotic extract of the respective one of the eight comparative strains prepared in section 1 of “Experimental Materials” of this example was dissolved in an appropriate amount of dimethyl sulfoxide (DMSO), so as to obtain a test solution having a concentration of 10 mg/mL, which served as a test solution of a respective one of an experimental group and comparative groups 1 to 9 as shown in Table 5 below.

TABLE 5

| Group | Ingredient in test solution |
|---|---|
| Experimental | Postbiotic extract of the present disclosure |
| group | |
| Comparative | Protein extract of Lactobacillus plantarum |
| group 1 | CB102 |
| Comparative | Postbiotic extract of Lactobacillus |
| group 2 | acidophilus JCM1132 |
| Comparative | Postbiotic extract of Lactobacillus casei |
| group 3 | JCM1134 |
| Comparative | Postbiotic extract of Limosilactobacillus |
| group 4 | fermentum CECT5716 |
| Comparative | Postbiotic extract of Lactobacillus |
| group 5 | rhamnosus GG |
| Comparative | Postbiotic extract of Bifidobacterium bifidum |
| group 6 | JCM1255 |
| Comparative | Postbiotic extract of Bifidobacterium lactis |
| group 7 | JCM10602 |
| Comparative | Postbiotic extract of Bifidobacterium longum |
| group 8 | CB108 |
| Comparative | Postbiotic extract of Bacillus coagulans |
| group 9 | CB85 |

### Experimental Procedures:

Determination of the activity of xanthine oxidase (XO) was performed with primarily reference to the method described in Hudaib M. M. et al. (2011), Pharmacogn. Mag., 7(28):320-324. In brief, 2 μL of the test solution of each group was added with 798 μL of 0.2 M sodium pyrophosphate buffer (with a pH value of 7.5) and 0.1 U xanthine oxidase (XO), followed by uniformly mixing at 37° C. for 5 minutes, so as to obtain a mixture of each group. Subsequently, the mixture of each group was added with 200 μL of 0.6 mM xanthine, followed by uniformly oscillating, so as to obtain a test sample of each group. In addition, the procedures and conditions for preparing a test sample of control group were similar to those of the experimental group, except that in the control group, 2 μL of the test solution including the postbiotic extract of the present disclosure of the experimental group was replaced by 2 μL of allopurinol.

Thereafter, the test sample of each group was subjected to determination of absorbance at a wavelength of 295 nm (OD₂₉₅) using an UV/VIS spectrophotometer (Manufacturer: Chrom Tech, Model no.: CT-2200).

The xanthine oxidase inhibition rate (%) of each group was calculated using the following Equation (1):

$$A = [ 1 - ( B / C ) ] \times 100 \qquad ( 1 )$$

where A=xanthine oxidase inhibition rate (%)
   B=OD₂₉₅ value determined in test sample of each group
   C=OD₂₉₅ value determined in test sample of control group

### Results:

TABLE 6

| | Xanthine oxidase |
|---|---|
| Group | inhibition rate (%) |
| Experimental group | 71 |
| Comparative group 1 | 15 |
| Comparative group 2 | 37 |
| Comparative group 3 | 33 |
| Comparative group 4 | 39 |
| Comparative group 5 | 40 |
| Comparative group 6 | 28 |
| Comparative group 7 | 25 |
| Comparative group 8 | 22 |
| Comparative group 9 | 19 |

Table 6 shows the xanthine oxidase inhibition rate determined in each group. As shown in Table 6, the xanthine oxidase inhibition rate determined in the experimental group was substantially higher than that determined in the comparative group 1, indicating that the postbiotic extract of Lactobacillus plantarum CB102, which was prepared by subjecting the protein extract of Lactobacillus plantarum CB102 to the hydrolysis treatment using the glutamic acid protease, could substantially exert an excellent effect on inhibiting activity of xanthine oxidase. In addition, the xanthine oxidase inhibition rate determined in the experimental group was substantially higher than those determined in the comparative groups 2 to 9, demonstrating that the postbiotic extract of Lactobacillus plantarum CB102 could effectively reduce uric acid production in vitro, and such effect is substantially better than those of other eight LAB strains.

### Example 4. Evaluation of the Effect of Postbiotic Extract of Lactobacillus plantarum CB102 on Alleviating Hyperuricemia

In this example, potassium oxonate (PO) and adenine were used to induce hyperuricemia in male ICR mice, so as to evaluate the effect of the postbiotic extract of the present disclosure on alleviating hyperuricemia.

### Experimental Materials:

### 1. Preparation of Solution of Postbiotic Extract of the Present Invention

Briefly, an appropriate amount of the postbiotic extract of the present disclosure prepared in Example 1 was formulated in an appropriate amount of sterile water, so as to prepare the solution of the postbiotic extract of the present invention having a concentration of 10 mg/ml.

### 2. Preparation of Potassium Oxonate (PO) Solution

Briefly, an appropriate amount of potassium oxonate (PO, Manufacturer: MedChemExpress, Cat. no.: HY-17511) was dissolved in an appropriate amount of phosphate buffered saline (PBS), so as to prepare the potassium oxonate (PO) solution having a concentration of 20 mg/mL.

### 3. Preparation of Adenine Solution

Briefly, an appropriate amount of adenine (Manufacturer: Sigma-Aldrich, Cat. no.: A8626) was dissolved in an appropriate amount of sterile water, so as to prepare the adenine solution having a concentration of 10 mg/mL.

### Experimental Procedures:

### A. Administration of Postbiotic Extract of Present Disclosure and Induction of Hyperuricemia

First, the male ICR mice as described in section 3 of “General Experimental Materials” were randomly divided into 3 groups (n=8 mice in each group), including a normal control group, a pathological control group, and an experimental group. Next, the mice in the experimental group were fed, via oral gavage, with the solution of the postbiotic extract of the present invention prepared in section 1 of “Experimental Materials” of this example at a dose of 105 mg/kg of body weight, and the mice in each of the normal control group and the pathological control group were fed, via oral gavage, with equal volumes of sterile water. Each mouse was fed once daily for a total period of 35 days.

Starting on 15th day of administration of the Lactobacillus plantarum CB102 postbiotic extract solution or the sterile water, before each daily administration, each mouse in the pathological control group and the experimental group was intraperitoneally injected with the PO solution prepared in section 2 of “Experimental Materials” of this example at a dose of 300 mg/kg of body weight once daily, and was fed, via oral gavage, with the adenine solution prepared in section 3 of “Experimental Materials” of this example at a dose of 75 mg/kg of body weight every other day, and this treatment (i.e., the use of the PO and adenine to induce hyperuricemia) was continued until the end of the 35-day experimental period, so as to induce hyperuricemia. In addition, each mouse in the normal control group received no treatment.

### B. Determination of Blood Uric Acid Level

On the 1^{st}, 15^{th}, 21^{st}, 28^{th}, and 35^{th} days after the start of administration, blood samples were collected from facial veins of the mice of each group via puncture, followed by subjecting the thus collected blood samples to a centrifugation treatment at 3,000 rpm and 4° C. for 15 minutes, so as to obtain the resultant supernatants which served as serum samples. Thereafter, each serum sample was diluted 10-fold with PBS, and then was subjected to determination of blood uric acid level using a Mouse Uric Acid ELISA Kit (Manufacturer: MyBioSource, Inc., Cat. no.: MBS3805644) in accordance with the manufacturer's instructions.

The data thus obtained were analyzed according to the procedures as described in section 1 of “General Procedures”.

### C. Evaluation of Biochemical Markers for Kidney Function in Blood

The serum samples prepared on the 35^{th} day after the start of administration as described in section B of “Experimental Procedures” of this example were subjected to determination of blood urea nitrogen (BUN) and blood creatinine (Cr) levels using a Mouse Blood Urea Nitrogen ELISA Kit (Manufacturer: MyBioSource, Inc., Cat. no.: MBS2611085) and a Mouse Creatinine Assay Kit (Manufacturer: MyBioSource, Inc., Cat. no.: MBS763433), respectively, in accordance with the manufacturer's instructions.

The data thus obtained were analyzed according to the procedures as described in section 1 of “General Procedures”.

### D. Determination of Activities of Xanthine Oxidase (XO) in Blood and Liver

On the 35^{th} day after the start of administration, the mice in each group were sacrificed by CO₂ asphyxiation, and liver tissue was obtained from each mouse carcass. Next, the respective liver tissue was subjected to a homogenization treatment using a sonicator (Manufacturer: SCICO, Model no.: SC-HM100), followed by adding an appropriate amount of sterile water to the resultant homogeneous substance, thereby obtaining a liver sample having a concentration of 10 mg/mL. The thus obtained liver samples of the mice of each group and the serum samples prepared on the 35^{th} day after the start of administration as described in section B of “Experimental Procedures” of this example were subjected to determination of activity of xanthine oxidase (XO) using a Xanthine Oxidase Activity Colorimetric/Fluorometric Assay Kit (Manufacturer: BioVision, Cat. no.: K710-100) in accordance with the manufacturer's instructions.

The data thus obtained were analyzed according to the procedures as described in section 1 of “General Procedures”.

### Results:

### A. Determination of Blood Uric Acid Level

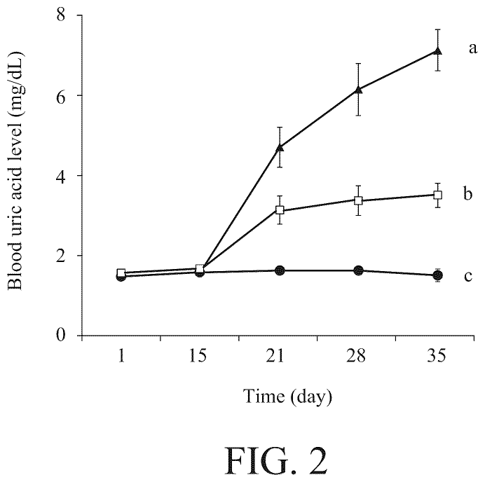

FIG. 2 shows the change in blood uric acid level determined over time in each group of mice. As shown in FIG. 2, from 15^{th} day onward after the start of administration (i.e., following the intraperitoneal injection of the PO solution and administration of the adenine solution), the blood uric acid level determined in the pathological control group significantly increased over time, while the blood uric acid level determined in the normal control group showed no significant change over time, indicating that the PO and adenine successfully induced hyperuricemia which led to a massive increase in blood uric acid in the mice of the pathological control group. In addition, from the 15^{th} day to 35^{th} day after the start of administration, compared with the pathological control group, a rise in the blood uric acid level determined in the experimental group was significantly diminished. These results demonstrate that the postbiotic extract of the present disclosure can effectively reduce the blood uric acid level in the hyperuricemia mice.

### B. Evaluation of Biochemical Markers for Kidney Function in Blood

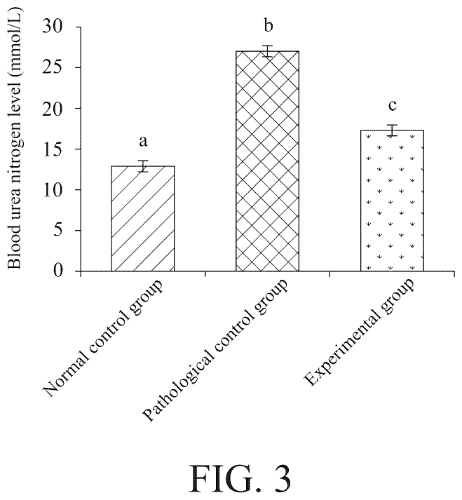

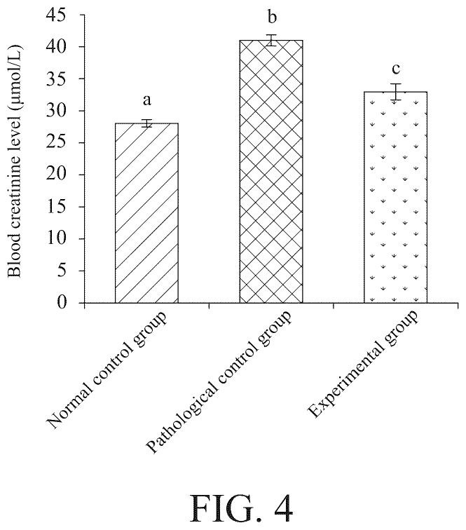

FIGS. 3 and 4 show the blood urea nitrogen (BUN) and blood creatinine (Cr) levels determined in each group of mice, respectively. As shown in FIGS. 3 and 4, compared with the normal control group, both the BUN and blood Cr levels determined in the pathological control group showed significant increases, indicating that the PO and adenine successfully induced hyperuricemia by impairing kidney filtration overtime, which in turn contributed to accumulation of blood uremic toxins in the mice of the pathological control group. In addition, compared with the pathological control group, both the BUN and blood Cr levels determined in the experimental group showed significant decreases. These results demonstrate that the postbiotic extract of the present disclosure can effectively alleviate the accumulation of the blood uremic toxins in the hyperuricemia mice.

### C. Determination of Activities of Xanthine Oxidase (XO) in Blood and Liver

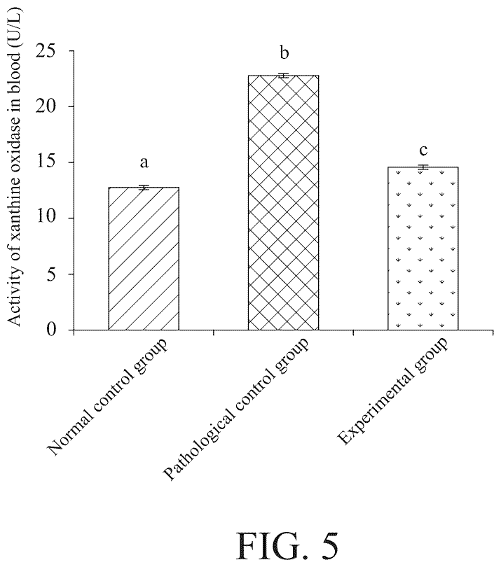

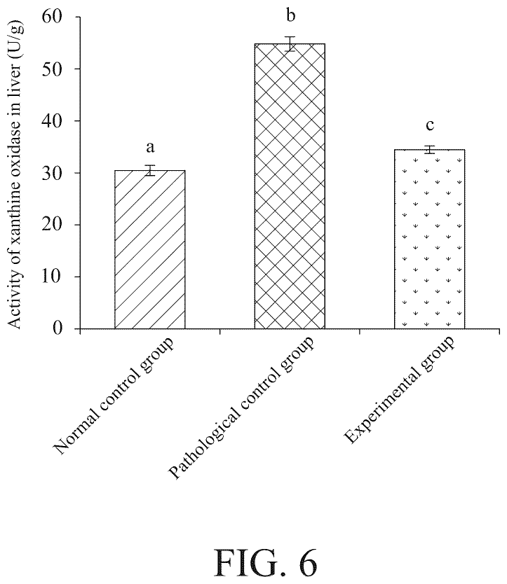

FIGS. 5 and 6 show the activities of xanthine oxidase (XO) in blood and liver determined in each group of mice, respectively. As shown in FIGS. 5 and 6, compared with the normal control group, the activities of XO in both blood and liver determined in the pathological control group showed significant increases, indicating that the PO and adenine successfully induced hyperuricemia by elevating the activities of XO in the blood and liver, which in turn contributed to a massive production of uric acid in the blood and liver of the mice in the pathological control group. In addition, compared with the pathological control group, the activities of XO in both the blood and liver determined in the experimental group showed significant decreases. These results demonstrate that the postbiotic extract of the present disclosure can effectively reduce the uric acid production in the blood and liver of the hyperuricemia mice by virtue of inhibiting the activities of XO in the blood and liver.

Summarizing the above test results, it is clear that the postbiotic extract of Lactobacillus plantarum CB102 of the present disclosure is capable of effectively inhibiting the activities of XO in the blood and liver, reducing the blood uric acid level, alleviating the accumulation of the blood uremic toxins, and reducing the uric acid production in the liver, and hence has a potential to be used in the preparation of a product for alleviating a uric acid metabolism-associated disorder (e.g., hyperuricemia).

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, the one or more features may be singled out and practiced alone without the another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A postbiotic extract of Lactobacillus plantarum CB102, which is prepared by the steps of:
subjecting the Lactobacillus plantarum CB102 to a protein extraction treatment, so as to obtain a protein extract; and
subjecting the protein extract to a hydrolysis treatment with a protease, so as to obtain the postbiotic extract of Lactobacillus plantarum CB102;
wherein the Lactobacillus plantarum CB102 is deposited at the Leibniz Institute DSMZ-German Collection of Microorganisms and Cell Cultures GmbH under an accession number DSM 33894 in accordance with the Budapest Treaty.

2. The postbiotic extract of Lactobacillus plantarum CB102 as claimed in claim 1, wherein the protease is selected from the group consisting of a glutamic acid protease, a serine protease, a glutamyl endopeptidase, an exopeptidase, and combinations thereof.

3. The postbiotic extract of Lactobacillus plantarum CB102 as claimed in claim 2, wherein the protease is the glutamic acid protease.

4. The postbiotic extract of Lactobacillus plantarum CB102 as claimed in claim 1, wherein a weight ratio of the protein extract to the protease ranges from 1.0:0.001 to 1.0:0.1.

5. A method for reducing a blood uric acid level, comprising administering to a subject in need thereof a composition including the postbiotic extract of Lactobacillus plantarum CB102 as claimed in claim 1.

6. The method as claimed in claim 5, wherein the composition is a food product or a pharmaceutical composition.

7. The method as claimed in claim 6, wherein the pharmaceutical composition is administered by a route selected from the group consisting of oral administration and parenteral administration.

8. A method for alleviating a uric acid metabolism-associated disorder, comprising administering to a subject in need thereof a composition including the postbiotic extract of Lactobacillus plantarum CB102 as claimed in claim 1.

9. The method as claimed in claim 8, wherein the uric acid metabolism-associated disorder is selected from the group consisting of gout, hyperuricemia, gouty arthritis, kidney disease, urolithiasis, nephrolithiasis, and combinations thereof.

10. The method as claimed in claim 8, wherein the composition is a food product or a pharmaceutical composition.

11. The method as claimed in claim 10, wherein the pharmaceutical composition is administered by a route selected from the group consisting of oral administration and parenteral administration.

12. A method for preparing a postbiotic extract of Lactobacillus plantarum CB102, comprising:
subjecting the Lactobacillus plantarum CB102 to a protein extraction treatment, so as to obtain a protein extract, and
subjecting the protein extract to a hydrolysis treatment with a protease, so as to obtain the postbiotic extract of Lactobacillus plantarum CB102;
wherein the Lactobacillus plantarum CB102 is deposited at the Leibniz Institute DSMZ-German Collection of Microorganisms and Cell Cultures GmbH under an accession number DSM 33894 in accordance with the Budapest Treaty.

13. The method as claimed in claim 12, wherein the protease is selected from the group consisting of a glutamic acid protease, a serine protease, a glutamyl endopeptidase, an exopeptidase, and combinations thereof.

14. The method as claimed in claim 13, wherein the protease is the glutamic acid protease.

15. The method as claimed in claim 12, wherein a weight ratio of the protein extract to the protease ranges from 1.0:0.001 to 1.0:0.1.
